(19) United States
(12) Patent Application Publication
(10) Pub. No.: US 2009/0084160 A1
(43) Pub. Date: Apr. 2, 2009

(54) GAS MEASURING DEVICE AND METHOD OF MANUFACTURING THE SAME

(75) Inventor: L. Rodney Bristol, Chalfont, PA (US)

Correspondence Address: RICK COMOGLIO;SR. INTELLECTUAL PROPERTY COUNSEL, TYCO INTERNATIONAL LTD., ONE TOWN CENTER ROAD, BOCA RATON, FL, 33486, US

(73) Assignee: SCOTT TECHNOLOGIES, INC., BOCA RATON, FL (US)

(21) Appl. No.: 12/238,798

(22) Filed: Sep. 26, 2008

Related U.S. Application Data
(60) Provisional application No. 60/997,083, filed on Oct. 1, 2007.

(51) Int. Cl. G01N 33/00; B32B 37/00

(52) U.S. Cl. 73/31.05; 156/64; 156/60; 73/23.2

(57) ABSTRACT

A gas measuring device for measuring a presence of a target gas in a fluid medium includes a sensor case defining a sensor cavity extending from an open end of the sensor case, wherein the fluid medium flows into the sensor cavity through the open end. A sensor is positioned within the sensor cavity for sensing the presence of the target gas, wherein the sensor has a sensing element on one side of the sensor, and the sensing element is positioned within the sensor cavity such that the sensing element faces away from the open end.

This application claims priority to and the benefit of the filing date of U.S. Provisional Application No. 60/997,083 filed Oct. 1, 2007 for “GAS MEASURING DEVICE AND METHOD OF MANUFACTURING THE SAME,” the complete subject matter of which is hereby incorporated by reference in its entirety.

### BACKGROUND OF THE INVENTION

The subject matter herein relates generally to gas measuring devices and methods of manufacturing gas measuring devices.

Gas measuring devices having sensors that detect certain chemicals or gases in air are utilized in many applications. For example, the detection of noxious gases such as carbon monoxide, hydrogen sulfide, nitrogen oxides, and the like is desirable so that a signal can be generated indicating the presence of such gases. Appropriate steps can then be taken to mitigate their effect or to remove persons from the presence of the gases.

One type of gas measuring device used to detect gas presence is a metal oxide semiconductor to provide early warning of the development of an explosion hazard (e.g. escaping flammable gas) or the presence of toxic gases or vapors in ambient air. The device typically includes a sensor provided on a heated substrate and includes two metallic electrodes connected to the sensor. The presence of gas posing a hazard is detected by a sensible change in the resistance of the sensor by means of the electrodes that are incorporated in a suitable electric circuit.

The reactions that allow the detection of target gases normally involve the oxidation of the target gas at the semiconductor (oxide) surface and a change in the electrical properties of the material. However, conventional sensors may be impacted by changes in temperature or humidity. Maintaining a constant temperature of the sensing element has proven problematic. At least some known devices have overcome such problems by superheating the sensor and maintaining the sensor at a superheated temperature, such that the sensor is less effected by changes in temperature. However, maintaining the sensor at superheated temperatures requires more power to operate the device.

A need remains for a gas measuring device and sensor that may be manufactured and operated in a cost effective and reliable manner.

### BRIEF DESCRIPTION OF THE INVENTION

In one embodiment, a gas measuring device is provided for measuring a presence of a predetermined gas in a fluid medium. The gas measuring device includes a sensor case defining a sensor cavity extending from an open end of the sensor case, wherein the fluid medium flows into the sensor cavity through the open end. A sensor is positioned within the sensor cavity for sensing the presence of the predetermined gas, wherein the sensor has a sensing element on one side of the sensor, and the sensing element is positioned within the sensor cavity such that the sensing element faces away from the opening.

Optionally, a porous screen may cover the open end and allow the fluid medium to flow therethrough. The sensor case may include a bottom generally opposed to the open end, and the sensor is positioned within the sensor cavity such that the sensing element faces the bottom of the sensor case. The sensing element may be responsive to a predetermined gas such that the electrical properties of the sensing element vary based on a presence of the predetermined gas. Optionally, the gas measuring device may further include a controller operatively connected to the sensing element for measuring at least one electrical property of the sensing element. The sensor may include a substrate having the sensing element applied to one side of the substrate and a heating element applied to the opposite side of the substrate, wherein the heating element is capable of heating the sensing element to a predetermined temperature. The sensing element and the heating element may cover substantially similar areas of the substrate.

In another embodiment, a method of manufacturing a gas measuring device is provided wherein the method includes providing a sensor case defining a sensor cavity extending from an open end of the sensor case and optionally covering the open end of the sensor cavity with a porous screen. The method also includes positioning a sensor having a sensing element on one side of the sensor within the sensor cavity such that the sensing element faces away from the opening or optional screen. The sensor is configured for sensing a presence of a target gas flowing into the sensor cavity.

In a further embodiment, a method of manufacturing a gas sensor is provided that includes providing a substrate having a sensing element and a heating element. The heating element is capable of heating the sensing element to an operating temperature, and the heating element has a resistance characteristic. The method also includes calibrating the heating element by measuring an initial resistance of the heating element at a first temperature less than the operating temperature of the gas sensor, and then calculating an operating resistance of the heating element at the operating temperature.

### BRIEF DESCRIPTION OF THE DRAWINGS

FIG. 1 illustrates a gas measuring device having a sensor formed in accordance with an exemplary embodiment.

FIG. 2 illustrates a control circuit for the gas measuring device and sensor shown in FIG. 1.

FIG. 3 is a cross sectional view of an exemplary embodiment of the sensor for the gas measuring device shown in FIG. 1.

FIG. 4 is a flow chart for an exemplary method of manufacturing a gas measuring device, such as the gas measuring device shown in FIG. 1.

FIG. 5 is a flow chart for another exemplary method of manufacturing a gas measuring device, such as the gas measuring device shown in FIG. 1.

### DETAILED DESCRIPTION OF THE INVENTION

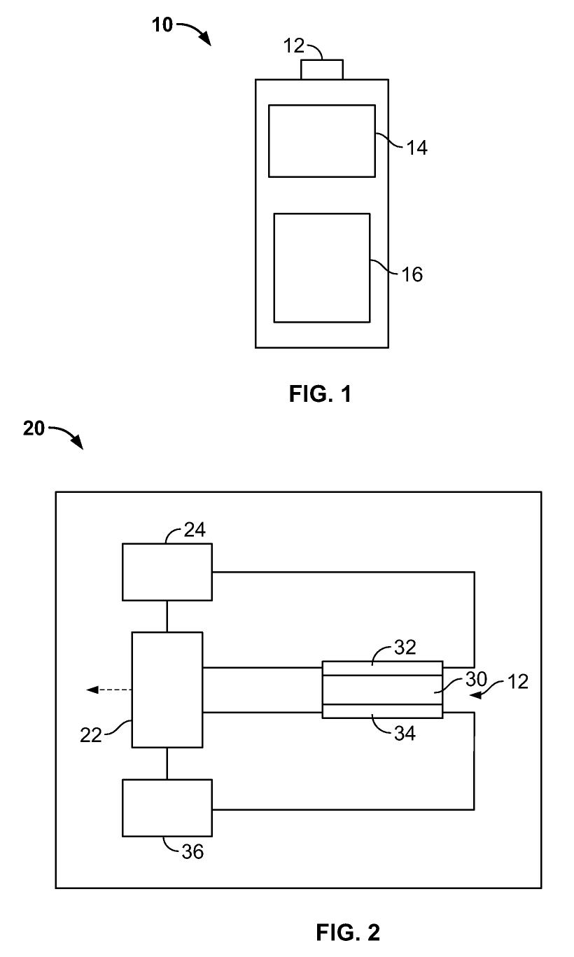

FIG. 1 illustrates a gas measuring device 10 having a sensor 12. The gas measuring device 10 is used for measuring a presence of a certain gas or gases in a fluid medium, such as air. The gas measuring device 10 may be used for measuring the presence of noxious, toxic, combustible or other harmful type of gas that may be present in the fluid medium. For example, the gas measuring device 10 may be used to detect for gases such as hydrogen sulfide, carbon monoxide, nitrogen oxides, and the like. In one embodiment, the gas measuring device 10 merely detects whether or not the gas is present. In other embodiments, the gas measuring device 10 may detect the amount or concentration of the gas when present.

The gas measuring device 10 communicates results, such as the concentration of the gas, by any known method, such as on a display 14. Optionally, a user interface 16, such as a keypad, may be provided for user interaction with the device 10. The gas measuring device 10 may alert the user as to the presence of a gas, or the presence of a gas above a threshold level, such as with an audible or visible alarm. In some embodiments, the gas measuring device 10 may communicate with other devices or systems to alert the device or system as to the presence of a gas. Such communication may be wired or wireless. The gas measuring device 10 may be portable and carried by a user, or alternatively, may be mounted to a structure in a desired location.

The sensor 12 is provided on the gas measuring device 10 such that the sensor 12 is exposed to the air surrounding the gas measuring device 10. While the sensor 12 is illustrated as being provided on an external surface of the gas measuring device 10, the sensor 12 may be internally housed within the gas measuring device 10 and air flow may be directed to the sensor 12, such as through a port open to the external environment or by air being pumped to the sensor 12.

FIG. 2 schematically illustrates a control circuit 20 for the gas measuring device 10 and sensor 12. The control circuit 20 includes a controller 22 that is operatively connected to the sensor 12. The control circuit 20 also includes a low voltage power supply 24 that is operatively connected to the sensor 12.

In an exemplary embodiment, the sensor 12 is a metal oxide sensor, however, other types of sensors may be used with the gas measuring device 10, and the metal oxide sensor illustrated in the figures is illustrative and is not intended to be limiting. The sensor 12 includes a substrate 30, such as a ceramic insulator. One example of a substrate 30 for the sensor 12 is an alumina substrate. The sensor 12 also includes a sensing element 32 on the substrate 30 and a heating element 34 on the substrate 30. In an exemplary embodiment, the sensing element 32 includes a gas sensitive material and is applied to one side of the substrate 30. The heating element 34 is applied to the opposite side of the substrate 30. In an alternative embodiment, the sensing element 32 and the heating element 34 may be applied to the same side of the substrate 30. Optionally, the sensor 12 may be a layered structure, wherein the sensing element 32 and/or the heating element 34 are layers applied to the substrate 30. For example, the sensing element 32 may be a film material screen printed on the substrate 30. The sensing element 32 may be a porous nanostructure. Similarly, the heating element 34 may be a film material screen printed on the substrate 30. The heating element 34 may be a ceramic micromachined heater.

The low voltage power supply 24 is operatively connected to the sensing element 32 and supplies a predetermined voltage to the sensing element 32. Optionally, the voltage supplied may be a constant voltage. The controller 22 is also connected to the sensing element 32 and measures at least one electrical property of the sensing element 32, such as a resistance, a conductance, a capacitance, and/or an impedance. The sensing element 32 is manufactured using a material having electrical properties that are affected by the presence of a predetermined target gas. For example, the sensing element 32 may be responsive to the gas such that the electrical properties of the sensing element 32 vary based on the presence and/or concentration of the gas. In an exemplary embodiment, gas adsorption on the surface of the sensing element 32 causes a change in electrical properties of the sensing element 32, such as a change in resistance. The changes in the electrical properties are detected and/or measured by the controller 22.

In an exemplary embodiment, the gas reactions with the sensing element 32 occur when the sensing element 32 is at an elevated temperature. The heating element 34 is used to elevate the temperature of the sensing element 32 to a predetermined temperature. A heater power supply 36 is provided for supplying power to the heating element 34. The power supply 36 may be operatively controlled by the controller 22, for example by pulse modulation. The amount of power supplied, the duration of the pulse and the frequency of the pulses affect the temperature of the heating element 34, and thus the sensing element 32. For example, in operation, when the heating element 34 is powered, the temperature of the substrate 30 is elevated, which thus raises the temperature of the sensing element 32 to a predetermined level. When the temperature of the sensing element 32 is at a predetermined level, the sensing element 32 may react with the gas at the surface of the sensing element 32. In an exemplary embodiment, the controller 22 is also connected to the heating element 34 to measure an electrical property of the heating element 34, such as a resistance, a conductance, a capacitance, and/or an impedance. The power supply to the heating element 34 may be controlled based on the electrical properties of the heating element 34. For example, the temperature of the heating element 34 may be related to a measured property of the heating element 34, such as resistance. As such, the temperature of the heating element 34 may be calculated and/or changed based on a measured electrical property of the heating element 34, such as the resistance. The gas measuring device 10 may be manufactured and operated in a similar manner as the device and sensor illustrated and described in copending U.S. patent application titled “GAS MEASURING DEVICE AND METHOD OF OPERATING THE SAME”, filed concurrently herewith and incorporated by reference herein in its entirety.

In alternative embodiments, other control schemes other than pulse modulation may be implemented to control the power supply to the heating element 34. In another embodiment, the heater power supply voltage may be proportionally controlled to maintain a desired heater resistance, where heater resistance is calculated from the ratio of heater voltage and a measurement of heater current.

In operation, when the controller 22 detects the presence of the gas, the controller 22 may output a signal relating to such presence and/or the concentration of the gas. The signal output from the controller 22 may be used by the gas measuring device 10 to alert the user and/or to display information relating to the presence/concentration of gas. In an exemplary embodiment, the controller 22 may include circuitry or circuit components, such as an amplifier that manipulates the signal from the sensing element 32, and/or an analog-to-digital converter that manipulates the signal from the sensing element 32. The manipulated signal may be output from the controller 22, or otherwise used by the gas measuring device 10 to perform other functions of the gas measuring device 10, such as the alerting or displaying. While the controller 22 is illustrated as being a common controller 22 that is operatively connected to both the heating element 34 and the sensing element 32, the control circuit 20 may include more than one controller.

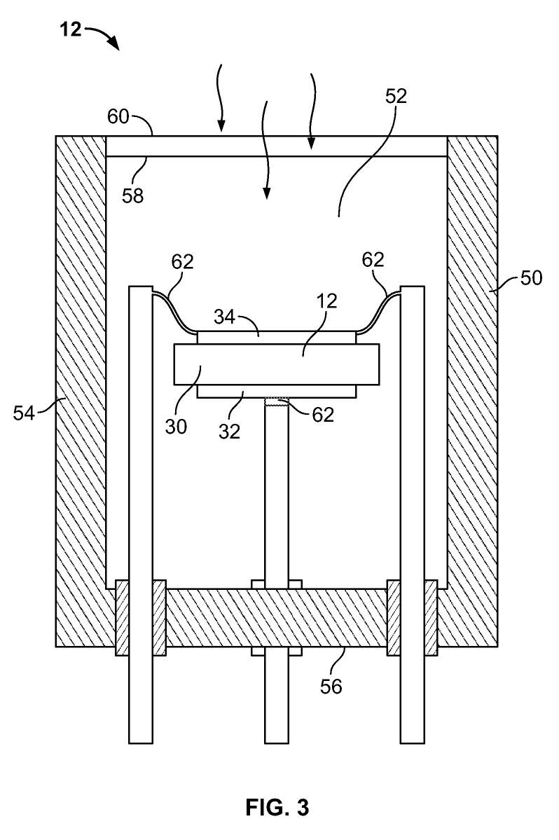

FIG. 3 is a cross sectional view of an exemplary embodiment of the sensor 12 for the gas measuring device 10 (shown in FIG. 1). The sensor 12 includes a sensor case 50 defining a sensor cavity 52. The sensor case 50 includes side walls 54 and a closed bottom 56. The sensor case 50 also includes an open top 58, generally opposite the bottom 56, and, optionally, a filter or screen element 60 at the open top 58. The screen element 60 is porous and allows air flow therethrough. Optionally, the screen element 60 may be used to filter certain gases from flowing therethrough. In an exemplary embodiment, the sensor case 50 may be generally cylindrical, but other shapes are possible in alternative embodiments.

The sensor 12 is positioned within the sensor cavity 52 for sensing the presence of predetermined target gases in the air flowing through the sensor cavity 52. Air from the external environment is able to flow through the screen element 60 and around the sensor 12. In an exemplary embodiment, the sensor 12 is positioned within the sensor cavity 52 such that the sensing element 32 faces away from the open top 58. For example, the sensing element 32 may generally face the bottom 56. Because the ambient environment is at a lower temperature than the sensor, and the open top 58 or, if it is used, the screen element 60, has a higher emissivity as compared to the sensor case 50, positioning the sensing element 32 away from the opening or the screen element 60 may reduce the rate or amount of reduction in temperature of the sensing element 32. As such, the temperature of the sensing element 32 may be more easily maintained at a constant temperature as compared to having the sensing element 32 face the ambient environment or the optional screen element 60. The sensor 12 may be positioned closer to the bottom 56 than the top 58 such that the sensing element 32 is proximate to the bottom 56 to efficiently retain the heat and thus maintain the temperature.

In an exemplary embodiment, the sensing element 32 and the heating element 34 each cover a predetermined amount of the substrate 30. Optionally, the sensing element 32 and the heating element 34 may cover substantially all of the substrate 30, however, the surface area of the sensing element 32 and the heating element 34 may be selected to cover only a select portion of the substrate 30. In one embodiment, the surface areas of the sensing element 32 and the heating element 34 may have substantially similar footprints (e.g. be substantially identically sized and shaped) and may be aligned with one another. As such, the heating element 34 may uniformly heat the sensing element 32. Optionally, the heating element 34 may have a larger footprint than the sensing element 32 such that the heating element 34 entirely covers the sensing element 32. For example, the perimeter of the sensing element 32 fits within the perimeter of the heating element 34.

A plurality of electrodes 62 are connected to the sensor 12. The electrodes 62 extend through the sensor case 50 into the sensor cavity 52. In an exemplary embodiment, the electrodes 62 are part of the control circuit 20 and connect the sensor 12 with the controller 22 (shown in FIG. 2) and/or the power supplies 24 and 36. Optionally, some electrodes 62 are connected to the heating element 34 and some electrodes 62 are connected to the sensing element 32.

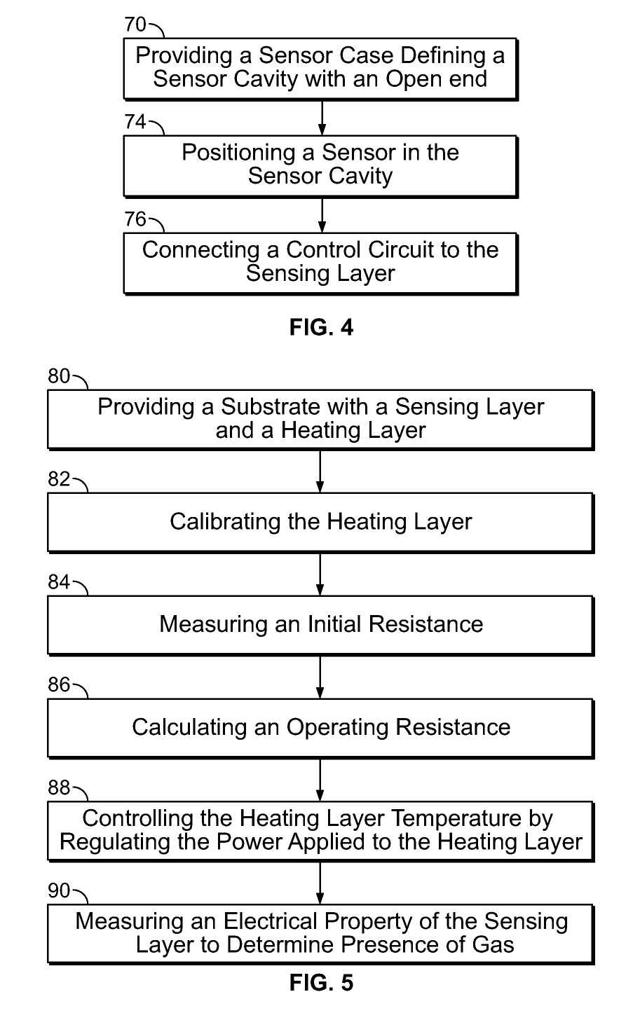

FIG. 4 is a flow chart illustrating an exemplary method of manufacturing a gas measuring device, such as the gas measuring device 10 (shown in FIG. 1). The method includes providing 70 a sensor case defining a sensor cavity extending from an open end of the sensor case. Optionally, the open end of the sensor cavity may be covered with a porous screen. The method also includes positioning 74 a sensor within the sensor cavity. The sensor may have a sensing element on one side of the sensor such that the sensing element faces away from the screen, and the sensor may be configured for sensing a presence of a predetermined target gas flowing into the sensor cavity through the porous screen. Other sensor types may be provided in alternative embodiments.

The sensor may be positioned within the sensor cavity such that the sensing element faces a closed bottom of the sensor case that is generally opposed to the open face. The sensor may have a sensing element that is responsive to a predetermined target gas such that the electrical properties of the sensing element vary based on a presence of the predetermined target gas. The sensor may include a substrate having the sensing element applied to one side of the substrate and a heating element applied to the opposite side of the substrate, wherein the heating element is capable of heating the sensing element to a predetermined temperature.

The method may also include the step of connecting 76 a controller to the sensing element for measuring at least one electrical property of the sensing element. In operation, the controller is used to monitor or measure at least one electrical property of the sensing element to detect the presence of at least one gas of interest in the air. In operation, the controller is also used to control the temperature of the heating element. For example, the controller may include a heater power supply that is operatively coupled to the heating element. The controller supplies power to the heating element according to a control scheme for elevating the temperature of the heating element and the sensing element. Optionally, the controller may monitor or measure at least one electrical property of the heating element to control the power supply to the heating element.

FIG. 5 is a flow chart illustrating another exemplary method of manufacturing a gas measuring device, such as the gas measuring device 10 (shown in FIG. 1). The method includes providing 80 a substrate having a sensing element and a heating element, wherein the heating element is capable of heating the sensing element to a predetermined operating temperature and wherein the heating element has a resistance characteristic that may be different from one sensor to another. The resistance characteristic may vary from one sensor to another as directly or indirectly affected by such properties as thickness, bulk resistivity, and surface area of the sensing element. Rather than tightly controlling the resistance characteristic, such as by trimming the sensing element or controlling the size of the sensing element by a costly application or manufacturing step, the system is configured to accommodate variations in resistance characteristics between successive devices by calibrating the device to the particular sensor. The method includes calibrating 82 the heating element by measuring 84 an initial resistance of the heating element at a first temperature less than the operating temperature of the gas sensor, and then calculating 86 an operating resistance of the heating element at the operating temperature. In common practice according to prior art, an operating temperature of the sensing element is approximately determined by providing a predetermined heating element resistance and connecting a predetermined power supply operated at a predetermined operating point, such as 28 Ohms, to the heating element. Tightly controlling properties to achieve a predetermined resistance may incur significant costs in fabrication. Additionally, trimming the heating element to obtain a predetermined resistance may impair the thermal coupling between the heating element and sensing element, which may impair the accuracy of the temperature of the sensing element. For example, the sensing element may have a different shape than the heating element after trimming which may affect the thermal gradient of the sensing element, such as heating the sensing element non-uniformly across the surface area thereof.

The method may include the step of connecting the heating element to a controller and controlling 88 the heating element temperature by regulating the power applied to the heating element as needed to maintain the calculated operating temperature. The controller measures a resistance of the heating element and the controller is configured to electrically power the heating element to increase the temperature of the heating element until the resistance is at the operating resistance. The controller may control the temperature of the heating element. The method may also include the step of connecting the sensing element to a controller and measuring 90 an electrical property of the sensing element to determine the presence of a target gas. The sensing element is responsive to a predetermined target gas such that the electrical properties of the sensing element vary based on a presence of the predetermined gas. The controller may measure the electrical properties of the sensing element.

During operation of the gas sensor, an electrical property of the heating element, such as the resistance, is measured by the controller. The resistance of the heating element may be affected by the size, surface area, and/or volume of the heating element, and as such, the gas sensor may need to be calibrated as the size, surface area, and/or volume of the heating element may be different for different gas sensors. The electrical properties of the heating element are also affected by the temperature of the heating element, and as such, the gas sensor may have a desired operating temperature. In an exemplary embodiment, the heating element has a given electrical property characteristic that can be used to compare the electrical property versus the temperature. For example, the characteristic may be a substantially linear characteristic, a non-linear characteristic, a substantially logarithmic characteristic, and the like. When the temperature is increased, the resistance may vary according to the characteristic, and similarly, when the temperature is decreased, the resistance may vary according to the characteristic. As such, when the initial resistance at a known initial temperature (e.g. room temperature) is measured, the operating resistance for the heating element may be calculated based on a desired operating temperature. For example, if the desired operating temperature of the heating element is 350 degrees, then the operating resistance may be calculated based on the characteristic of resistance versus temperature. As such, during operation, the temperature of the heating element may be controlled by using the controller to measure the resistance of the heating element, and either increasing or decreasing the power applied to the heating element based on the measured resistance.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112, sixth paragraph, unless and until such claim limitations expressly use the phrase “means for” followed by a statement of function void of further structure.

What is claimed is:

1. A gas measuring device for measuring a presence of a target gas in a fluid medium, the gas measuring device comprising:
a sensor case defining a sensor cavity extending from an open end of the sensor case, wherein the fluid medium flows into the sensor cavity through the open end; and
a sensor positioned within the sensor cavity for sensing the presence of the target gas, the sensor having a sensing element on one side of the sensor, the sensing element being positioned within the sensor cavity such that the sensing element faces away from the opening.

2. The gas measuring device of claim 1, wherein the sensor case includes a bottom generally opposed to the open end, the sensor being positioned within the sensor cavity such that the sensing element faces the bottom of the sensor case.

3. The gas measuring device of claim 1, wherein the sensing element is responsive to a target gas such that the electrical properties of the sensing element vary based on a presence of the target gas.

4. The gas measuring device of claim 1, further comprising a circuit operatively connected to the sensing element for measuring at least one electrical property of the sensing element.

5. The gas measuring device of claim 1, wherein the sensor includes a substrate having the sensing element applied to one side of the substrate and a heating element applied to the opposite side of the substrate, the heating element being capable of heating the sensing element to a predetermined temperature.

6. The gas measuring device of claim 1, wherein the sensor includes a substrate having the sensing element applied to one side of the substrate and a heating element applied to the opposite side of the substrate, wherein the sensing element and the heating element cover substantially similar areas of the substrate.

7. The gas measuring device of claim 1, wherein the sensor includes a substrate having the sensing element applied to one side of the substrate and a heating element applied to the opposite side of the substrate, wherein the heating element covers at least as much of the substrate as the sensing element.

8. The gas measuring device of claim 1, wherein the sensor includes a substrate, the sensing element and the heating element being aligned with one another on opposite sides of the substrate such that a perimeter of the sensing element fits within a perimeter of the heating element.

9. A method of manufacturing a gas measuring device comprising:
providing a sensor case defining a sensor cavity extending from an open end of the sensor case; and
positioning a sensor having a sensing element on one side of the sensor within the sensor cavity such that the sensing element faces away from the opening, wherein the sensor is configured for sensing a presence of a target gas flowing into the sensor cavity.

10. The method of claim 9, wherein the positioning further comprises positioning the sensor within the sensor cavity such that the sensing element faces a closed bottom of the sensor case that is generally opposed to the opening.

11. The method of claim 9, wherein the positioning further comprises positioning a sensor having a substrate having the sensing element applied to one side of the substrate and a heating element applied to the opposite side of the substrate, the heating element being capable of heating the sensing element to a predetermined temperature.

12. The method of claim 9, wherein the positioning further comprises positioning a sensor having a substrate having the sensing element applied to one side of the substrate and a heating element applied to the opposite side of the substrate, wherein the sensing element and the heating element cover substantially similar areas of the substrate.

13. A method of manufacturing gas sensors comprising:
providing a substrate having a sensing element and a heating element, wherein the heating element is capable of heating the sensing element to an operating temperature, the heating element having a predetermined resistance characteristic; and
calibrating the heating element by measuring an initial resistance of the heating element at a first temperature less than the operating temperature of the gas sensor, and then calculating an operating resistance of the heating element at the operating temperature.

14. The method of claim 13, wherein the heating element has a surface area and the resistance characteristic is based on the surface area, the calibrating is performed to allow for variations in the surface area of the heating element.

15. The method of claim 13, wherein the providing a substrate having a sensing element includes applying a sensing element to a surface of the substrate, wherein the sensing elements of successive gas sensors that have different resistance characteristics are operated at different operating resistances.

16. The method of claim 13, wherein, when the measured initial resistance of the heating element of successive gas sensors is different, the calculated operating resistance of the respective heating elements is different.

17. The method of claim 13, further comprising connecting the heating element to a controller, wherein the controller measures a resistance of the heating element and the controller is configured to electrically power the heating element to increase the temperature of the heating element until the resistance is at the operating resistance.

18. The method of claim 13, further comprising controlling the heating element temperature by regulating the power applied to the heating element as needed to maintain the calculated operating temperature.

19. The method of claim 13, further comprising connecting the sensing element to a controller, and measuring a property of the sensing element to determine the presence of a target gas.

20. The method of claim 13, further comprising connecting the sensing element to a controller, wherein the sensing element is responsive to a target gas such that the electrical properties of the sensing element vary based on a presence of the target gas, and wherein the controller measures the electrical properties of the sensing element.
